(19) United States
(12) Patent Application Publication
(10) Pub. No.: US 2011/0288521 A1
(43) Pub. Date: Nov. 24, 2011

(54) INTRADERMAL INJECTOR AND USES THEREOF

(75) Inventor: John W. Bingham, Elizabeth, CO (US)

(73) Assignee: Pharma Jet ,Inc.

(21) Appl. No.: 12/921,375

(22) PCT Filed: Mar. 9, 2009
(86) PCT No.: PCT/US2009/036560

§ 371 Date: Dec. 14, 2010

Related U.S. Application Data
(60) Provisional application No. 61/034,919, filed on Mar. 7, 2008.

(51) Int. Cl. A61M 5/30

(52) U.S. Cl. 604/500; 604/68

(57) ABSTRACT

The invention provides for a needle-free or needle-less intradermal injection device that is capable of delivering an agent of interest to only the intradermal space. The intradermal device can deliver lower volumes of an agent than commonly used with present devices. In one aspect of the invention, the intradermal device is useful for delivering one or more agents to the intradermal space for eliciting immune responses particular to the dermal layer. In other aspects of the invention, the intradermal device is useful for delivering one or more agents to the intradermal space for treating, delaying development of delaying the progression of preventing, and/or ameliorating symptoms of various diseases, disease states, and conditions.

### CROSS-REFERENCES TO RELATED APPLICATIONS

This application claims priority benefit to provisional application 61/034,919, filed on Mar. 7, 2008, which is incorporated by reference herein in its entirety.

### FIELD OF THE INVENTION

The present invention relates to a needle-free or needle-less injector that can deliver a high-pressure jet of fluid, such as an agent or medicament, to specifically the intradermal layer of the skin in an individual. The present invention also relates to methods of delivering a specific dose of an agent or medicament via a needle-less injector to the intradermal space of an individual.

### BACKGROUND OF THE INVENTION

The advantages of needle-less injection devices have been recognized for some time. Some of these advantages include: the absence of needle stick injuries that present hazards to healthcare workers; a reduction in the risk of cross-contamination among patients, whether, human or animal; the elimination of needle breakage in the tissue of the human or animal; and that the jet of liquid medicament is generally smaller than the diameter of a hypodermic needle and thus may be less invasive than a hypodermic needle.

Because of the well-known advantages of needle-less injection, there are many different kinds of such devices, including pneumatic powered needle-less injection devices that are designed to provide multiple doses to patients or animals, or gas actuated, which are for single or multiple use. Most known needle-less injection devices operate by using a piston to drive the fluid to be delivered though a fine nozzle that creates a small, high pressure stream that penetrates the skin simply due to the high pressure. Multi-dose and single-dose devices depend on a source of energy to drive air or working fluid that is used to operate the piston that drives the fluid through the nozzle. Thus, a serious limitation of these devices is that they must have a readily available source of energy to drive the piston. This makes these devices impractical for use in hospitals and/or clinics, and in most field situations, especially in remote areas where access to dependable energy is uncertain.

These injector devices are also large, sometimes expensive units, and generally adapted to retain large quantities of medicament for repeated injections. Most of these machines are not portable and have historically been used chiefly for mass inoculation programs. Because of the disadvantages of injection devices that use high-pressure fluids to drive the piston and deliver multiple injections, a great deal of attention has been given to the development of a spring-powered needle-less injection device for delivering a single injection. The success of the known devices has been limited, due to problems associated with safety and reliability. The issues regarding safety generally involve the possibility of accidental discharge of the device and the possibility of transmitting diseases between patients due to carryover of body fluids. The problems associated with reliability generally involve the device's ability to deliver a full, known dose of the liquid.

There are also disadvantages related to the containment of the fluid formulations in single dose needle-less injectors. Individual doses of a liquid formulation can be delivered via the injector. However, often the volume of medicament held in the conventional injectors is too large, for example, when injecting an infant or small animal, such as a mouse. Often one-half or more of the dosage is not required and hence would be wasted or the injection could not be given safely to such patient because it is more invasive than necessary. This decreases the practicality and use of the injectors in certain environments.

Another disadvantage of known needle-less injectors is the inability to direct the location of the injection, i.e., intramuscularly, intradermally and/or subcutaneously. Other needle-free injections devices have been described in the art. See, for example, U.S. Pat. Nos. 5,899,879; 6,942,638; U.S. Publication Nos. 2007/0118094; 2007/0191762; 2007/0027428; and PCT/US2005/046041. However, the previously described devices deliver the agent of interest to multiple layers of the skin, e.g., intramuscularly, intradermally and/or subcutaneously. There is a lack of needle-free injection devices that are capable of delivering one or more agents of interest to only the intradermal space. In addition, there is a lack of needle-free injection devices and that are capable of delivering lower volume of one or more agents, with a short injection time, and/or with minimal pain to the recipient of the injection. The invention described herein fulfils these needs and provides additional benefits.

Throughout the specification, references are made to patents, patent applications and other references, all of which are hereby incorporated by reference in its entirety.

### BRIEF SUMMARY OF THE INVENTION

The invention provides for a needle-free intradermal injection device that is capable of delivering an agent of interest to only the intradermal space. In one aspect of the invention, the intradermal device can deliver lower volumes of an agent than commonly used with present devices. In some embodiments, the lower volume of an agent is 0.1 cc or 0.2 cc. In another aspect of the invention, the intradermal device is capable of delivering an agent to an individual in need thereof with an injection time of less than about 1 second. In another aspect of the invention, the intradermal device provides methods for delivering one or more agents to the intradermal space for eliciting immune responses particular to the dermal layer. In one aspect, the immune response is activating dendritic cells and/or antigen presenting cells residing in the dermal layer.

### BRIEF DESCRIPTION OF THE DRAWINGS

FIG. 1 depicts one cross section of the intradermal, needle-free device (upper panel) with another needle-free device (lower panel) that does not limit delivery of an agent to the dermal layer. The shaded area is the hammer.

FIGS. 2-6 depict the results of tolerance analysis of the syringe portion of the needle-free device.

FIG. 7 depicts a photograph of the present hand-held, needle-free injector device with the syringe separated from the injector portion of the system.

FIG. 8 depicts the results of intradermal delivery wherein the fraction of intradermal delivery was determined in a patient sample.

FIG. 9 depicts the results of a study done in human patients (cadavers) wherein 0.1 cc of Indian ink was administered using the intradermal device described herein. In this instance, the human was an 18 year old male with a body mass index (BMI) of 24.5.

FIG. 10 depicts the results of a study done in human patients (cadavers) wherein 0.2 cc of Indian ink was administered using the intradermal device described herein. In this instance, the human was a 20 year old female with a body mass index (BMI) of 23.5.

FIG. 11 depicts the results of a study done in human patients (cadavers) wherein 0.1 cc of Indian ink was administered using the intradermal device described herein. In this instance, the human was a 20 year old female with a body mass index (BMI) of 23.5.

FIG. 12 depicts the results of a study done in human patients (cadavers) wherein 0.5 cc of Indian ink was administered using the standard device that delivers agents to both the intradermal space as well as the subcutaneous (SC) space.

FIG. 13 is a photograph of a mouse study wherein the intradermal delivery device described herein was used and the dermal layer of the mouse is shown.

FIGS. 14-18 depict the results of rat studies wherein the intradermal delivery device described herein was used and the dermal layers were analyzed. FIG. 16 shows the results where two different volumes (0.1 cc and 0.2 cc) were administered intradermally.

FIG. 19 is a photograph of a human individual's arm after using the intradermal device described herein.

FIG. 20 depicts the results shown in a pig study where the numbers represent decreasing spring energy. This series is similar to a titration, with the right-most injection in the photograph being completely subcutaneous and the left most injection in the photograph being completely intradermal. The intervening injections are somewhere in between. These results demonstrate that Applicants can control the distribution of the injection and can deliver the agent at one or more levels to effectuate the response desired.

### DETAILED DESCRIPTION OF THE INVENTION

The present invention described herein provides for an improved device for intradermal delivery of an agent and methods of its use. Other needle-free injections devices have been described in the art. See, for example, U.S. Pat. Nos. 5,899,879; 6,942,638; U.S. Publication Nos. 2007/0118094; 2007/0191762; 2007/0027428; and PCT/US2005/046041. However, the present device of the invention differs from the previously described devices in that the present device has been manipulated in multiple parameters that allows for solely intradermal delivery. The previously described devices are fixed dose (e.g., 0.5 cc) injection systems that were designed to deliver intramuscular (IM) and subcutaneous (SC) injections.

### DEFINITIONS

As used herein, an “individual” is a vertebrate. In one aspect of the invention, the vertebrate is a mammal. An “individual” can be a human and at certain times, the individual is a patient. Vertebrates can also include, but are not limited to, farm or production animals (e.g., pigs, cattle, fowl, cows, horses), sport animals, pets, primates, mice and rats.

“Agent,” as used herein, can encompass any type of composition for delivery to an individual, including but not limited to, vaccines, medicaments, immunomodulating compounds, immunostimulatory compounds, immunosuppressive compounds and the like. In some cases, the agent can be a test compound or a compound used for purposes of testing the delivery profile (e.g., Indian ink or saline). In some embodiments, the agent includes adjuvants and/or other pharmaceutically acceptable excipient and/or standard preservatives. It is to be understood that one or more agents can be administered to an individual.

An “intramuscular (IM) injection” is one that passes through the skin and subcutaneous tissue and penetrates the underlying skeletal muscle.

A “subcutaneous (SC) injection” is one that fully penetrates the skin and is retained in the space between the skin and the underlying musculature. In one embodiment of the invention, a subcutaneous injection is given in the fatty layer of tissue just under the skin.

An “intradermal (ID) injection” floods the epidermal and dermal layers with the agent being injected but does not travel as deep as a subcutaneous injection.

### Intradermal Device

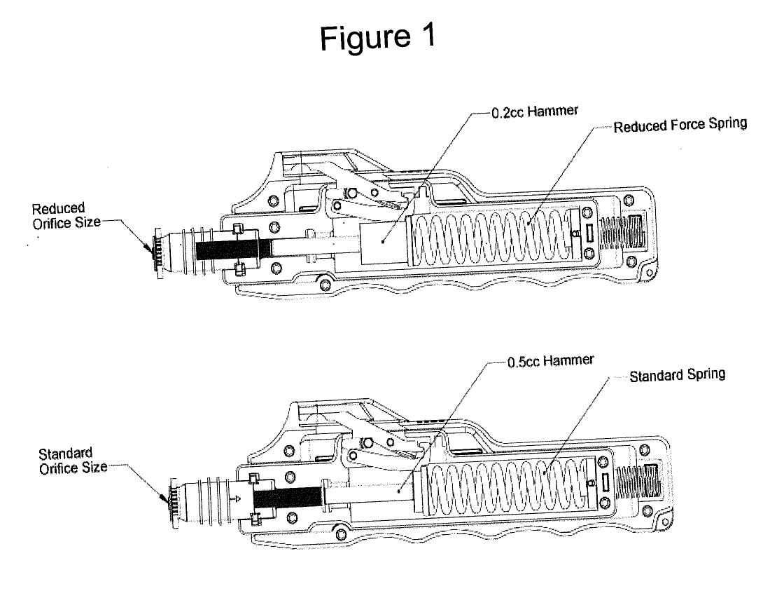

As depicted in the upper panel of FIG. 1, the present invention is, in one aspect, a hand-held device that is capable of delivering an agent to an individual in need thereof intradermally. Other methods of intradermal delivery is known in the art, however, they commonly use needles and require prolonged contact of the needle with the individual. The use of needles to deliver agents intradermally is not ideal, especially with young children, because of the need for the children to remain still to obtain the delivery to the dermal layer. In addition, delivery via the needle is dependent on the skill of the healthcare worker in getting it just under the dermal layer and slowly flooding the agent or medicament there. Statistically, very few healthcare workers can inject intradermally. Furthermore, for many individuals, there is pain and/or discomfort associated with needle delivery. The intradermal device described herein provides several advantages over existing needle-free jet delivery devices in that it is capable of delivering an agent to only the intradermal layer, is capable of delivering a precise and lower volume of an agent to the intradermal layer, imposes relatively less pain on the individual receiving the injection compared to other injection devices, and is capable of stimulating a immune response in the dermal layer, such as activating antigen presenting cells (APCs) and dendritic cells in the dermal layer. In addition, the intradermal device of the invention is capable of delivering one or more agent to an individual consistently, whereas delivery via a needle is frequently inconsistent because it is dependent on the skill of the healthcare worker in getting it just under the dermal layer and slowly flooding the agent or medicament there.

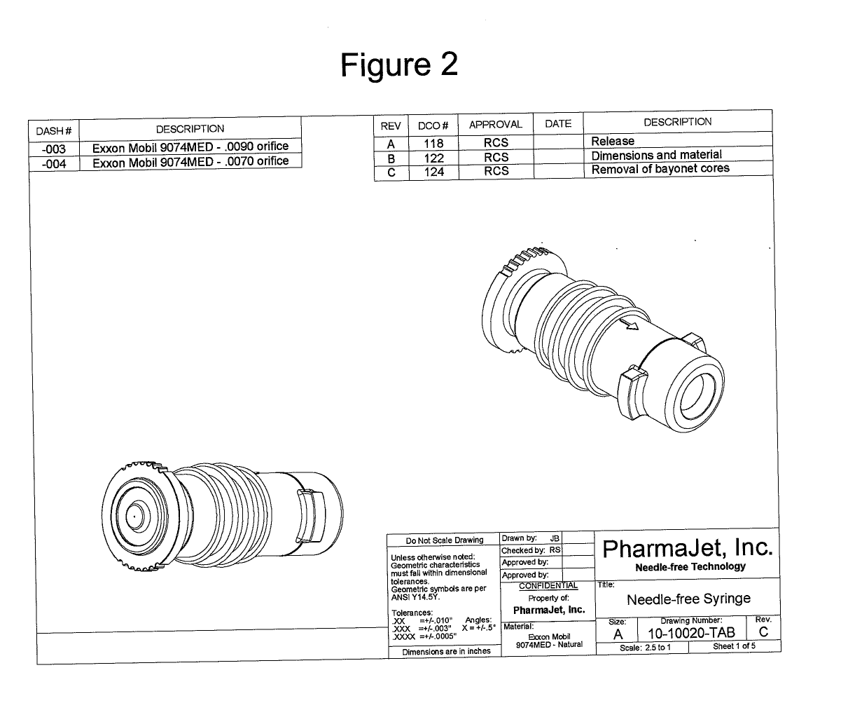

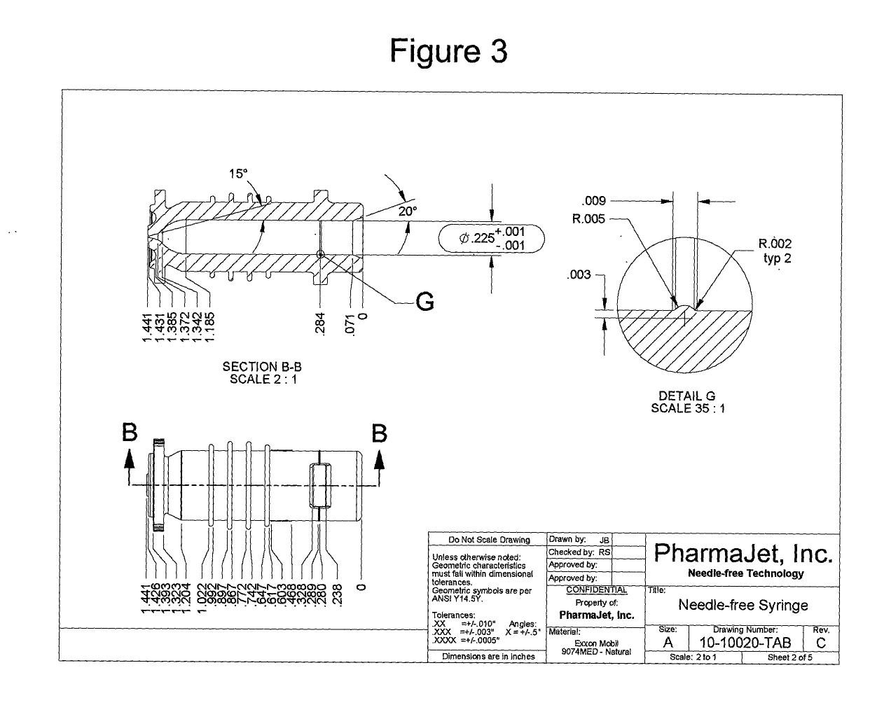

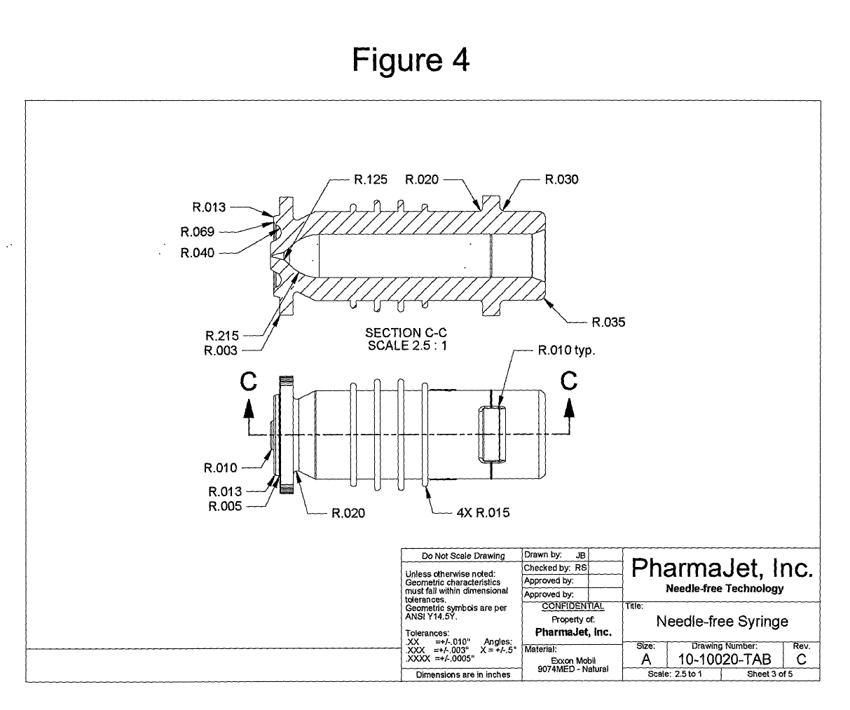

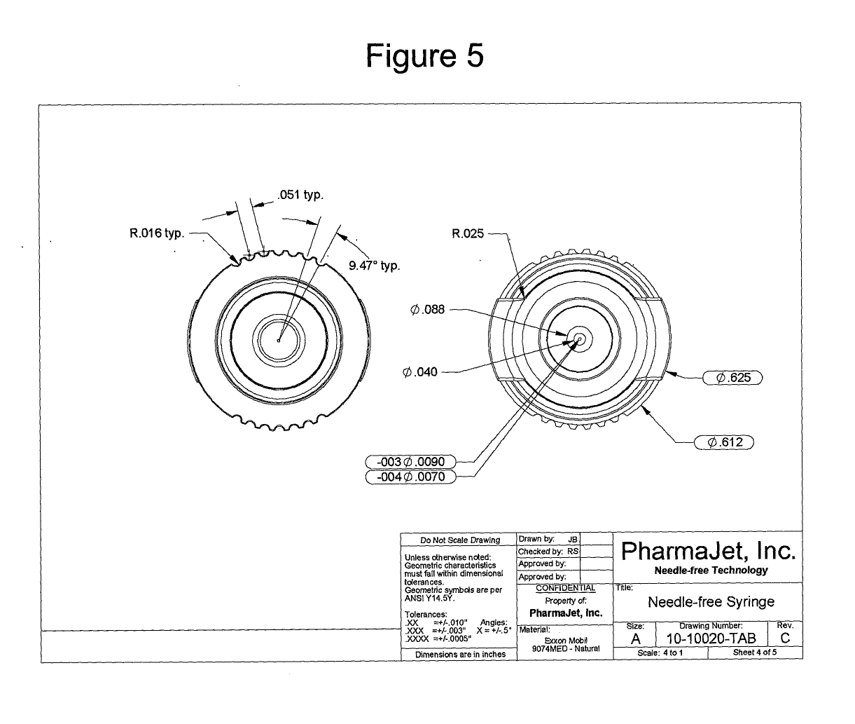

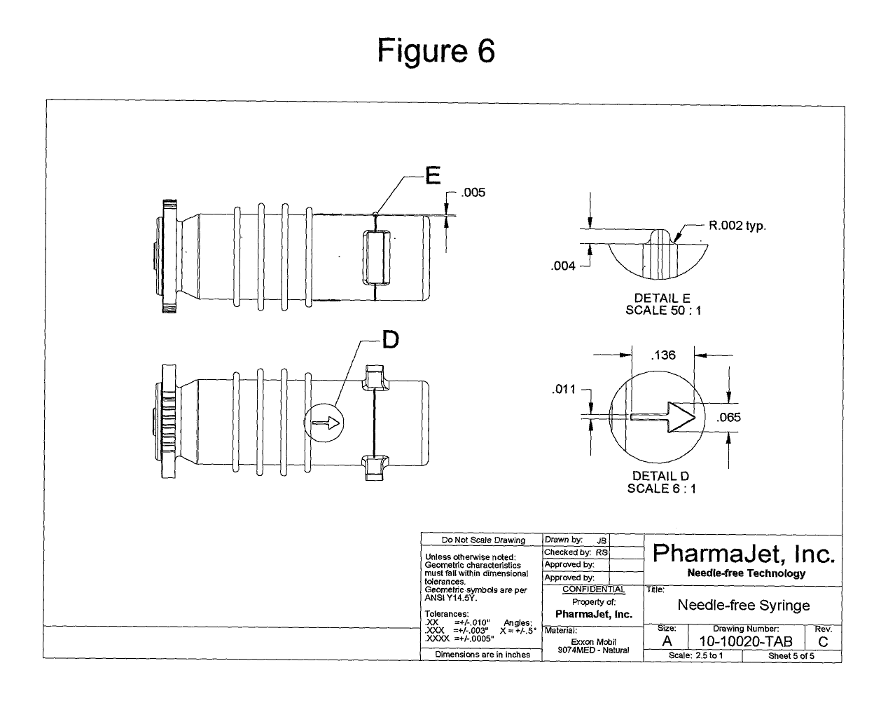

FIG. 1 depicts a cross-section of two devices and compares the intradermal device of the present invention (upper panel) with existing needle-free injection devices that deliver an agent to multiple layers (e.g., intradermal, subcutaneous and intramuscular) in an individual. The intradermal device is different from previously described needle-free injection devices in several ways. The intradermal device is made by changing several components to deliver a lower dose (e.g., 0.1 cc and 0.2 cc) of intradermal (ID) injection compared to previously described needle-free injection systems. By changing the hammer, main spring, and reducing the orifice size in the Syringe, the system can deliver the required volume to the ID space. FIG. 1-6 illustrate the various parameters that can be changed to achieve this result and, as such, is one embodiment of the invention that is contemplated. In addition, FIGS. 2-6 provides tolerance levels for the reduction in orifice size, the ranges of which are contemplated within the scope of the invention.

The hammer size is changed to allow for delivery of a volume of about 0.5 cc or lower. In one embodiment, as shown in FIG. 1, the shaded area is the hammer size, which has been reduced from 0.5 cc to 0.2 cc. The change in the hammer size allows for the delivery of a smaller volume of an agent (e.g., 0.2 cc). Similar changes can be effectuated to the hammer for fixed doses of between about 0.05 cc to about 0.5 cc, as discussed in greater detail below. This change in hammer size in combination with variations in force spring and variations in orifice size allows for the delivery of an agent to solely the intradermal space of an individual.

The spring force varies according to the individual to whom the injection is being administered. In some embodiments, it is an animal. Other factors for one of skill in the art to take into consideration are the size of the individual and/or tissue density. In one aspect of the invention, the spring force is about 35 pounds to about 130 pounds. In another aspect of the invention, it is between about 58 pounds to about 130 pounds. In another aspect of the invention, the spring force is about 35 pound to about 50 pounds. In another aspect of the invention, the spring force is about 35 pounds to about 75 pounds. In another aspect of the invention, the spring force is about 35 pounds to about 100 pounds. In another aspect of the invention, the spring force is about 35 pounds to about 75 pounds. In another aspect of the invention, the spring force is about 50 pounds to about 75 pounds. In another aspect of the invention, the spring force is about 50 pounds to about 130 pounds. In another aspect of the invention, the spring force is about 50 pounds to about 100 pounds. One of skill in the art can readily adjust the spring force to the amount necessary depending on the size of the individual and/or tissue density. For animals with thicker skin, such as water buffalo or cows, a greater spring force is used than for animals with thinner skin and lesser tissue density, such as humans. The skilled artisan can also rely on teachings in the art for guidance on aspects of an individual's skin. See, e.g., J. M. Waller and H. I. Maibach, Age and skin structure and function, a quantitative approach (I): Blood flow, pH, thickness, and ultrasound echogenicity, Skin Res Technol 11 (2005), p. 221; G. J. Fisher, The pathophysiology of photoaging of the skin, Cutis 75 (2005), pp. 5-8; E. Berardesca and H. Maibach, Ethnic skin: Overview of structure and function, J Am Acad Dermatol 48 (2003), pp. 139-142; S. Alaluf, D. Atkins and K. Barrett et al., Ethnic variation in melanin content and composition in photoexposed and photoprotected human skin, Pigment Cell Res 15 (2002), pp. 112-118; R. I. Kelly, R. Pearse and R. H. Bull et al., The effects of aging on the cutaneous microvasculature, J Am Acad Dermatol 33 (1995), pp. 749-756; J. W. Fluhr and P. M. Elias, Stratum corneum pH: formation and function of the ‘acid mantle’, Exogenous Dermatol 1 (2002), pp. 163-175; K. P. Wilhelm, A. B. Cua and H. I. Maibach, Skin aging Effect on transepidermal water loss, stratum corneum hydration, skin surface pH, and casual sebum content, Arch Dermatol 127 (1991), pp. 1806-1809; R. M. Lavker, P. S. Zheng and G. Dong, Aged skin: A study by light, transmission electron, and scanning electron microscopy, J Invest Dermatol 88 (1987) (suppl 3), pp. 44s-51s; M. Gniadecka, Effects of ageing on dermal echogenicity, Skin Res Technol 7 (2001), pp. 204-207; M. T. Hull and K. A. Warfel, Age-related changes in the cutaneous basal lamina: Scanning electron microscopic study, J Invest Dermatol 81 (1983), pp. 378-380; M. C. Branchet, S. Boisnic and C. Frances et al., Skin thickness changes in normal aging skin, Gerontology 36 (1990), pp. 28-35; and J. Sandby-Moller, T. Poulsen and H. C. Wulf, Epidermal thickness at different body sites: Relationship to age, gender, pigmentation, blood content, skin type and smoking habits, Acta Derm Venereol 83 (2003), pp. 410-413.

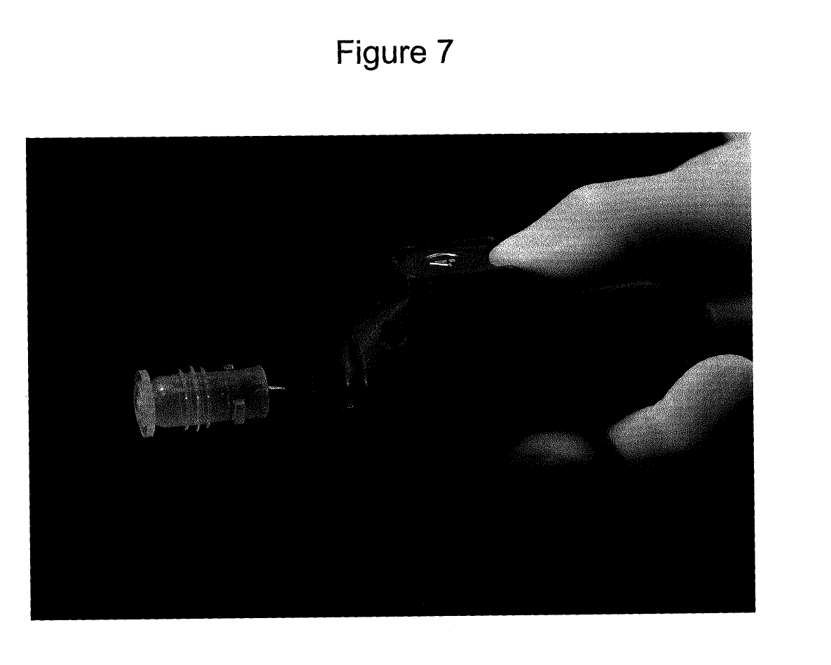

The orifice size can also be varied to achieve intradermal delivery. In one embodiment, the orifice size is 0.007 inch. In other embodiments, the orifice size is adjusted plus and/or minus the tolerance levels as indicated in FIGS. 2-6. In addition, FIGS. 1-6 provide additional guidance for the parameters that one of skill could use to in order to achieve one embodiment of the intradermal device. FIG. 7 is photograph showing the intradermal device.

In one embodiment, the orifice material is medical grade polypropylene (natural). In a preferred embodiment, the use of regrind is not recommended. Accordingly, the use of virgin material for the orifice is a more preferred embodiment. In addition, in other embodiments, it is recommended that no surface particulates are allowed on the orifice on the part which is detectable with normal vision under ambient light conditions. Further recommendation that can be included in other embodiments of the invention are the following: (1) that all parts be free of foreign debris; (2) the maximum parting line flash is 0.003″; (3) the maximum flash allowed on the bore be 0.0005″; (4) no visible knit line deformation of bore is allowed (5) maximum gate protrusion to be 0.003″; (6) surface finish should meet or exceed SPI B1 on cylinder exterior and entire bore; and (7) no mold release or plasticizers are allowed in manufacturing of this part.

Some advantages of the intradermal device are that it allows for efficient delivery of a small volume of an agent to the ID space in a short period of time (less than about 1 second, which is a shorter period than the human nervous system can respond) and with minimal pain to the recipient of the injection. In one aspect of the invention, the intradermal device delivers an agent in about 0.1 second. In other aspects of the invention, the intradermal device delivers an agent in about 0.2 second. In other aspects of the invention, the intradermal device delivers an agent in short period of time of about 0.1 to about 0.2 second. In other aspects of the invention, the intradermal device delivers an agent in short period of time of about 0.2 to about 0.3 second. In other aspects of the invention, the intradermal device delivers an agent in short period of time of about 0.3 to about 0.4 second. In other aspects of the invention, the intradermal device delivers an agent in short period of time of about 0.4 to about 0.5 second. In other aspects of the invention, the intradermal device delivers an agent in short period of time of about 0.5 to about 0.6 second. In other aspects of the invention, the intradermal device delivers an agent in short period of time of about 0.6 to about 0.7 second. In other aspects of the invention, the intradermal device delivers an agent in short period of time of about 0.7 to about 0.8 second. In other aspects of the invention, the intradermal device delivers an agent in short period of time of about 0.8 to about 0.9 second. In other aspects of the invention, the intradermal device delivers an agent in short period of time of about 0.9 to about 1 second. In other aspects of the invention, the intradermal device delivers an agent in short period of time of about 0.1 to about 1 second. In other aspects of the invention, the intradermal device delivers an agent in short period of time of about 0.1 to about 0.5 second. In other aspects of the invention, the intradermal device delivers an agent in short period of time of about 0.1 to about 0.3 second. In other aspects of the invention, the intradermal device delivers an agent in short period of time of about 0.1 to about 0.9 second.

The lowered volume that can be delivered saves on costs for vaccines since lesser amount has to be manufactured, delivered to the appropriate location around the globe and delivered to the individuals. The volume that can be delivered can range between about 0.05 cc to about 0.5 cc. In one aspect of the invention, the volume that can be delivered to the ID space is 0.1 cc. In another aspect of the invention, the volume that can be delivered to the ID space is 0.2 cc. In yet other aspects of the invention, the volume that can be delivered to the ID space is 0.05 cc, 0.06 cc, 0.07 cc, 0.08 cc, or 0.09 cc. In other aspects of the invention, volume that can be delivered to the ID space is from about 0.1 cc to about 0.2 cc. In yet other aspects of the invention, the volume that can be delivered to the ID space is 0.11 cc, 0.12 cc, 0.13 cc, 0.14 cc, 0.15 cc, 0.16 cc, 0.17 cc, 0.018 cc, or 0.19 cc. In yet other aspects of the invention, the volume that can be delivered to the ID space is from about 0.2 cc to about 0.3 cc. In yet other aspects of the invention, the volume that can be delivered to the ID space is from about 0.3 cc to about 0.4 cc. In yet other aspects of the invention, the volume that can be delivered to the ID space is from about 0.4 cc to about 0.5 cc. The hammer size should be adjusted accordingly to account for the volume of agent to be delivered as described herein and in the Figures. Furthermore, one of skill in the art will appreciate that the volume of one or more agents to an individual can depend on the physical characteristics of the individual itself. In cases of larger animals, such as a cow or water buffalo, then a larger volume, such as 0.5 cc could be delivered intradermally. However, in a smaller animal such as a mouse or rat, delivery of volume of 0.5 cc may be too large of a volume to deliver and could result in multilayer delivery (e.g., SQ or IM) instead of solely intradermal delivery. Accordingly, one of ordinary skill in the art should take care to adjust the volume size according to the individual receiving the injection. This is within routine skills possessed by one of ordinary skill in the art.

### Methods of Use

Without being bound by theory, the intradermal device is useful for eliciting immune responses specific to the dermal layer, such as APCs and dendritic cells. One factor to take into consideration when using the intradermal device is the thickness of the individual's skin. If an individual's skin is thinner than normal, then appropriate adjustments should be made so that the injection does not penetrate deeper into the skin into the muscle, for example. The adjustments that can be made include, but are not limited to, varying the spring force, varying the volume of the agent being administered to the individual, and varying the hammer size accordingly. See, for example, Laurent et al., Vaccine 25:6423-6430 (2007). This is useful in generating an antibody response to the agent being administered. In some aspects of the invention, the agent being administered by the intradermal device is a vaccine for either treatment or prophylaxis (including delaying the development) of diseases and disease states. In general, the intradermal device of the invention can be used to administer any agent or a combination of agents where intradermal delivery is desirable. The Examples section describes results of studies done in mouse, rat, humans and pigs that show that the intradermal device can achieve delivery of a particular agent to only the dermal layer in the test subject.

The intradermal device described herein can be used for treating, delaying development of, delaying the progression of, preventing, and/or ameliorating symptoms of various diseases, disease states and conditions described herein. In addition, it can be used for delivering agents that are useful for the eradication of etiological causes of various diseases and disease states. Accordingly, in some aspects of the invention, the diseases are infectious diseases or viral diseases. In other aspects of the invention, the intradermal device can be used to administer agents for treating, delaying development of, delaying the progression of, preventing, and/or ameliorating symptoms of cancer, autoimmune diseases, or allergies. Non-limiting examples include as chicken pox, measles, influenza, common cold, gastrointestinal diseases, hemorrhagic fever, hepatitis A and B (and others), mumps, rubella, pertussis, diphtheria, yellow fever, dengue fever, West Nile, small pox, malaria, polio, anthrax, tetanus, pneumococcal, HPV, HIV, malaria, shingles, rabies, tuberculosis, measles/mumps/rubella (MMR), cancer vaccines, and epithelial growth hormones.

In other aspects of the invention, the intradermal device can be used to deliver anethesetic agents, such as lidocaine, marcaine and the like. In yet another aspect of the invention, the intradermal device can be used to deliver agents for cosmetic purposes, e.g., Botox. One of skill in the art will appreciate that Botox can also be administered using the intradermal device for therapeutic purposes to treat various type of dystonia and muscle spasms, strabismus (“crossed eyes”) and blepharospasm.

In another aspect, the intradermal device can be used to deliver prophylactic vaccines in developing countries for eradicating or decreasing infectious diseases. Non-limiting examples include as chicken pox, measles, influenza, common cold, gastrointestinal diseases, hemorrhagic fever, hepatitis A and B (and others), mumps, rubella, pertussis, diphtheria, yellow fever, dengue fever, West Nile, small pox, malaria, polio, anthrax, tetanus, pneumococcal, HPV, HIV, malaria, shingles, rabies, tuberculosis, and measles/mumps/rubella (MMR). In other aspects of the invention, the intradermal device is used for eliciting an immune response in the dermis of an individual. In other aspects of the invention, the intradermal device is used for delivering vaccines to farm animals, sport animals and pets. Non-limiting examples include dogs, cats, sheep, cattle, horses, fowl (e.g., chicken, ducks, geese, etc.).

The following Examples are provided to illustrate, but not to limit, the invention.

### EXAMPLES

### Example 1

### Intradermal Delivery Performance

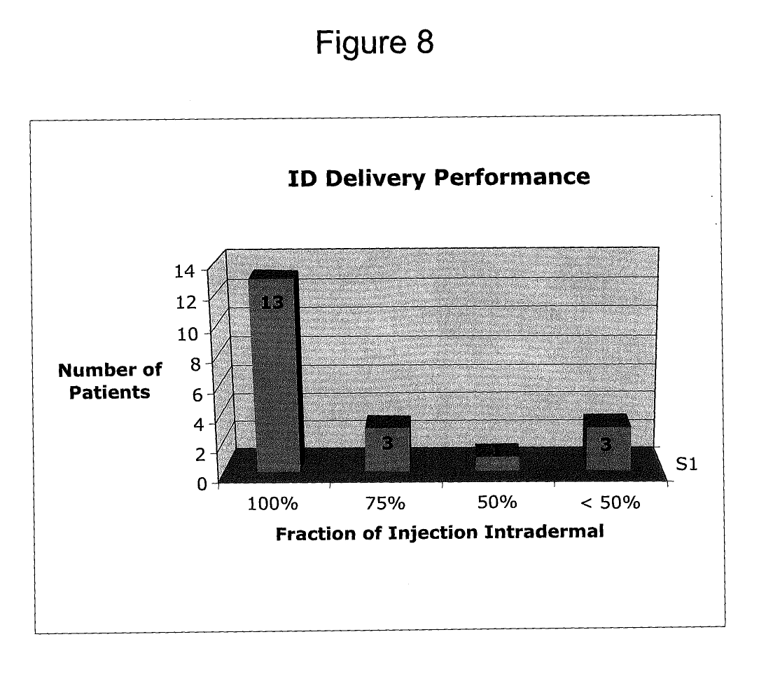

A total of 20 patients were tested for delivery performance of the intradermal device. As shown in FIG. 8, in 13 of the 20 patients, 100% of the injection was intradermal. In 3 of the 20 patients, 75% of the injection was intradermal. In the balance of the patients, intradermal delivery was achieved, but some of the injection went into deeper tissue, as follows: In 1 of 20 patients, about 50% of the injection was intradermal. Finally, in 3 of the 20 patients, less than 50% of the injection was intradermal. It is to be noted that one of the patients who showed less than 50% intradermal delivery had undergone extreme weight loss and had very thin skin so the intradermal delivery penetrated further than would have been expected in a person who had average skin thickness.

### Example 2

### Intradermal Delivery in Humans

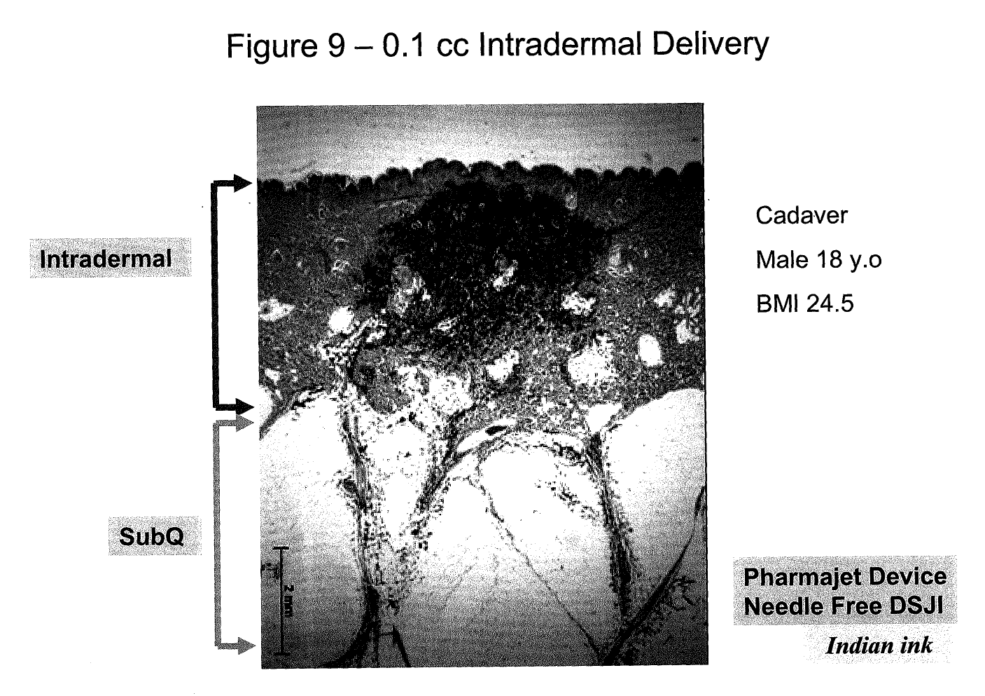

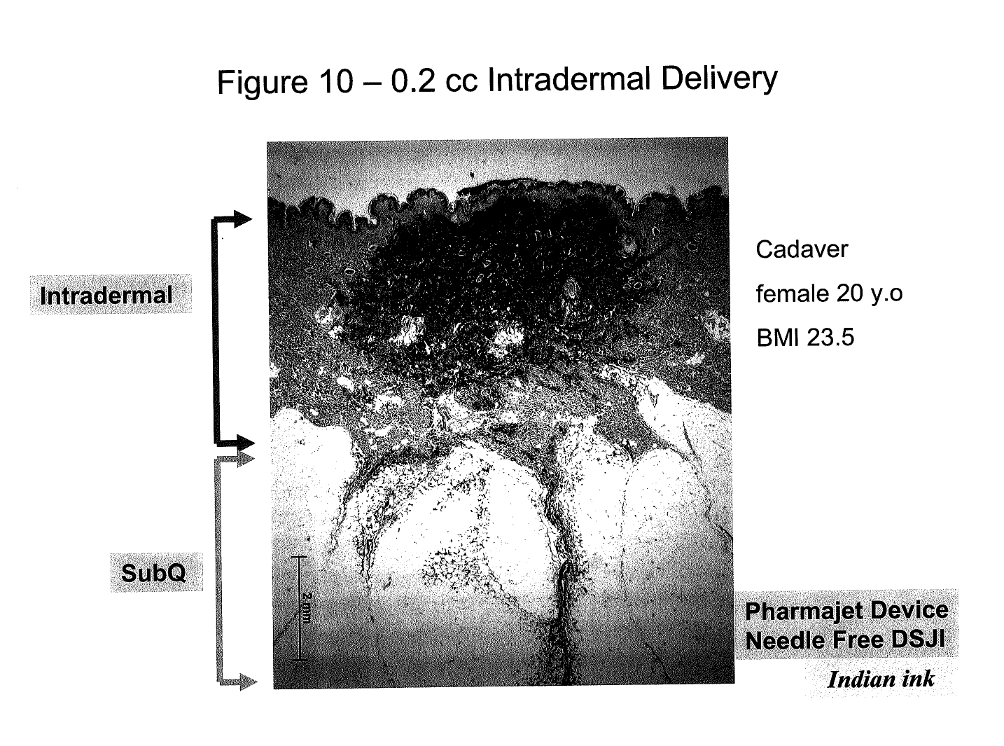

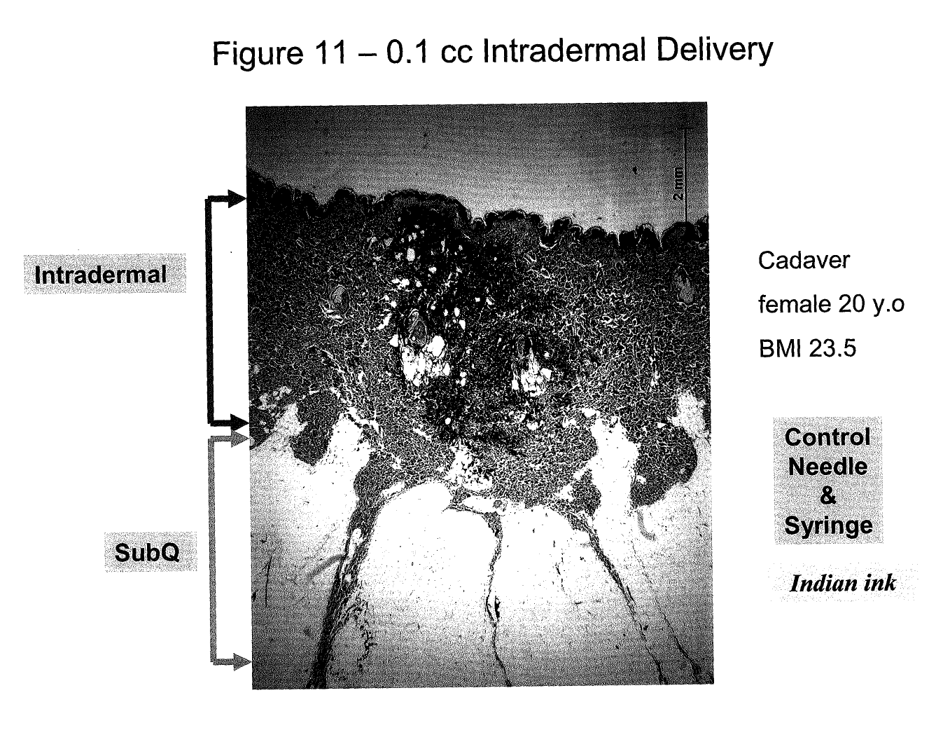

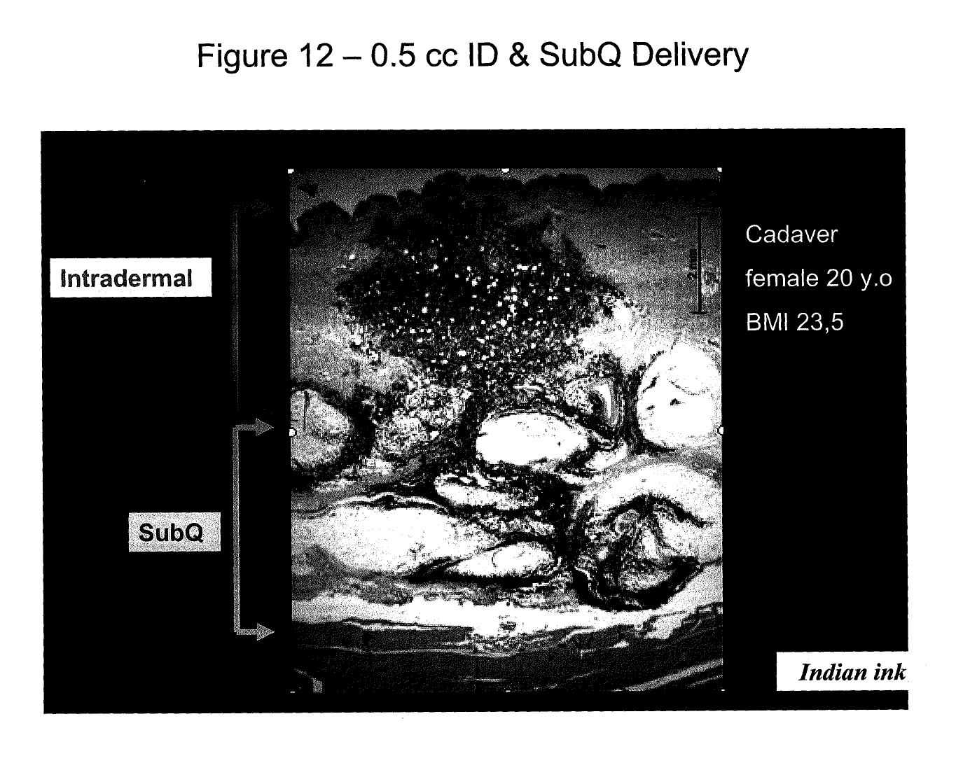

Human cadavers, both male and female, were used for testing of various volumes of intradermal delivery. FIGS. 9 and 11 show the results of 0.1 cc delivered intradermally with a test agent, Indian ink, and show that the intradermal device was successful in delivering the test agent to the ID space of the human skin. Similarly, 0.2 cc of the same test agent was administered to another human cadaver and the results are shown in FIG. 10. The results depicted therein also show the successful delivery of the test agent to the ID space. In contrast, other needle-free delivery devices which have not been adjusted as described herein to delivery solely to the ID space yield delivery to both ID and SC space, as shown in FIG. 12.

### Example 3

### Intradermal Delivery in Mice

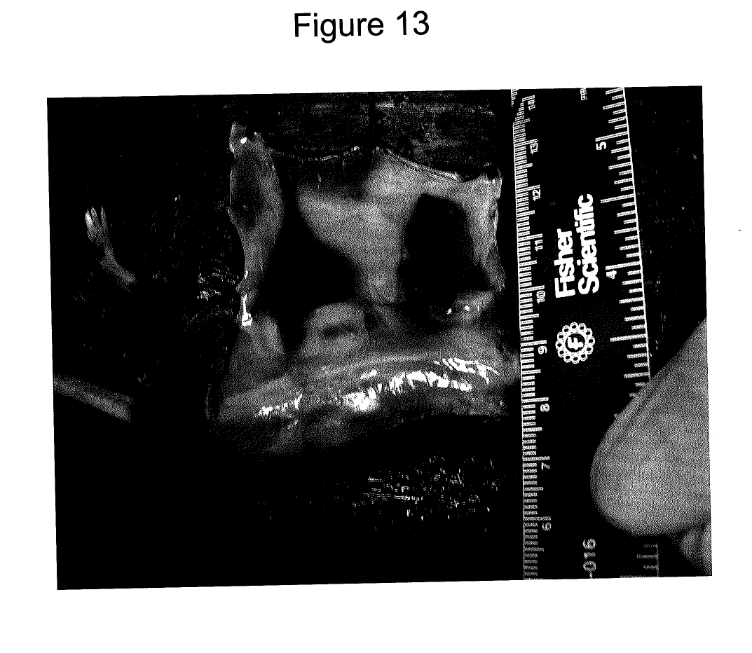

Mouse studies were conducted with a test agent, Indian ink, using the intradermal device described herein. As can be seen in FIG. 13, the delivery of the test agent was limited to just the dermal layer of the mouse skin.

### Example 4

### Intradermal Delivery in Rats

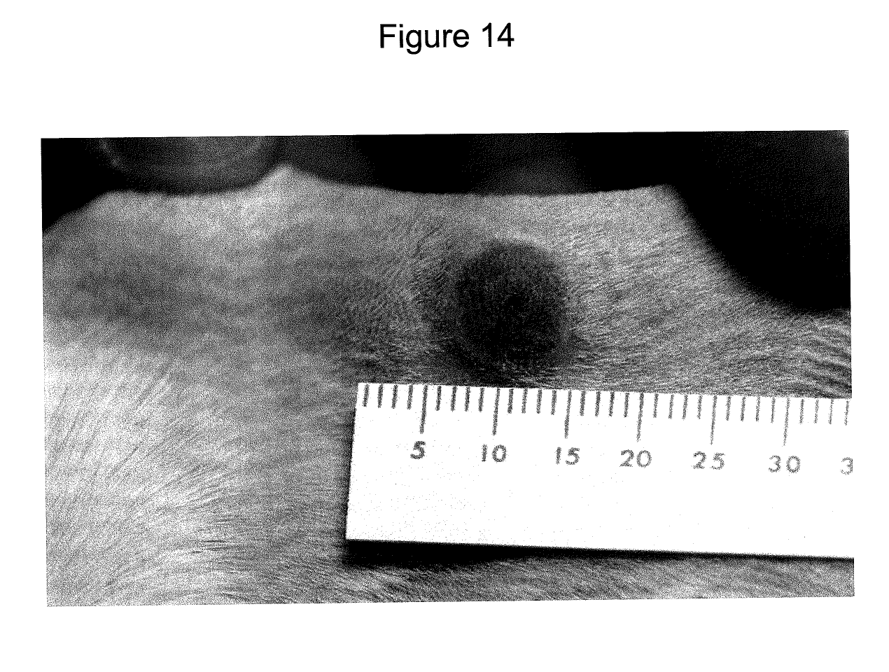

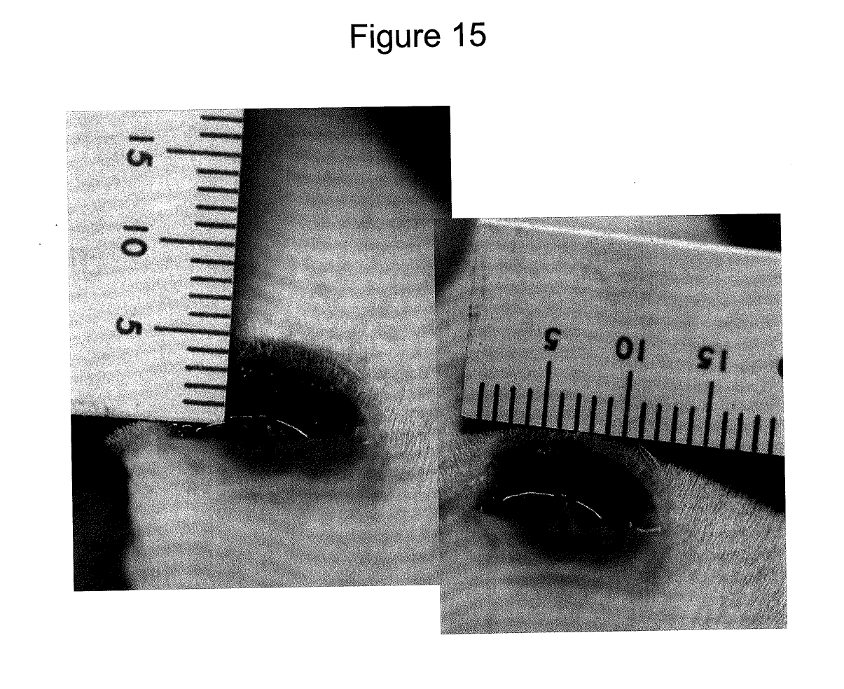

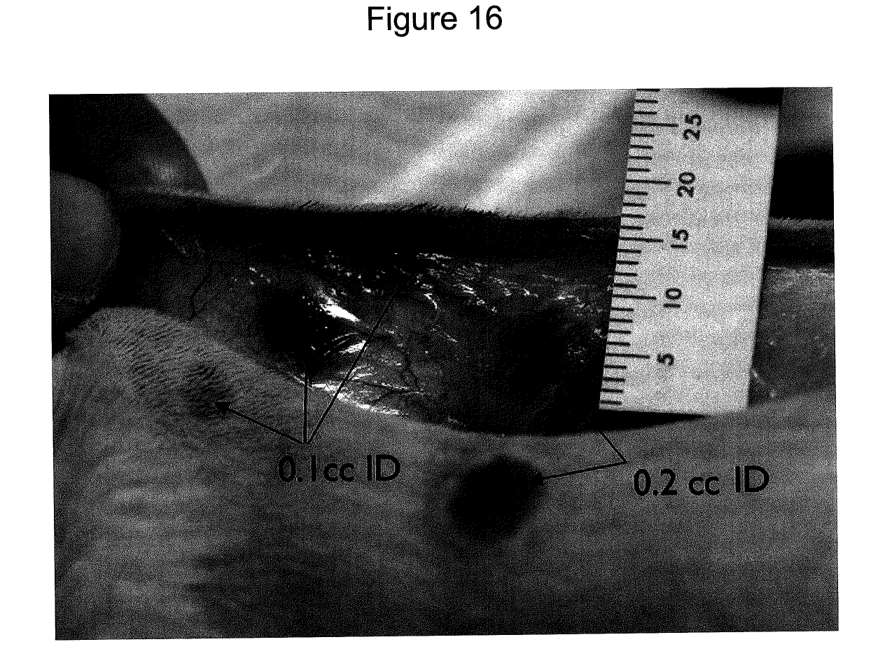

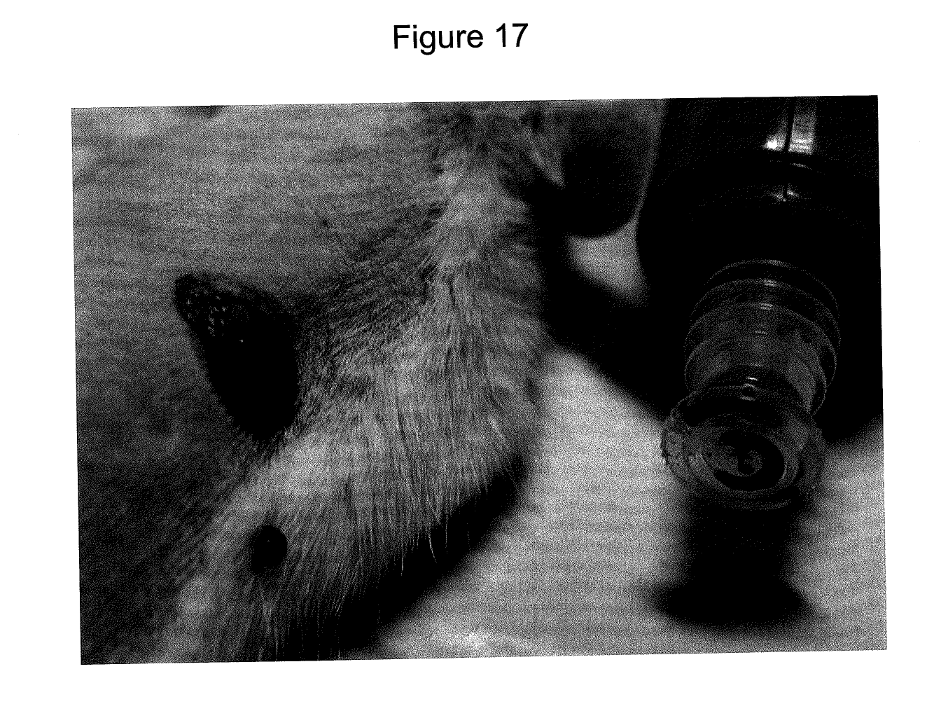

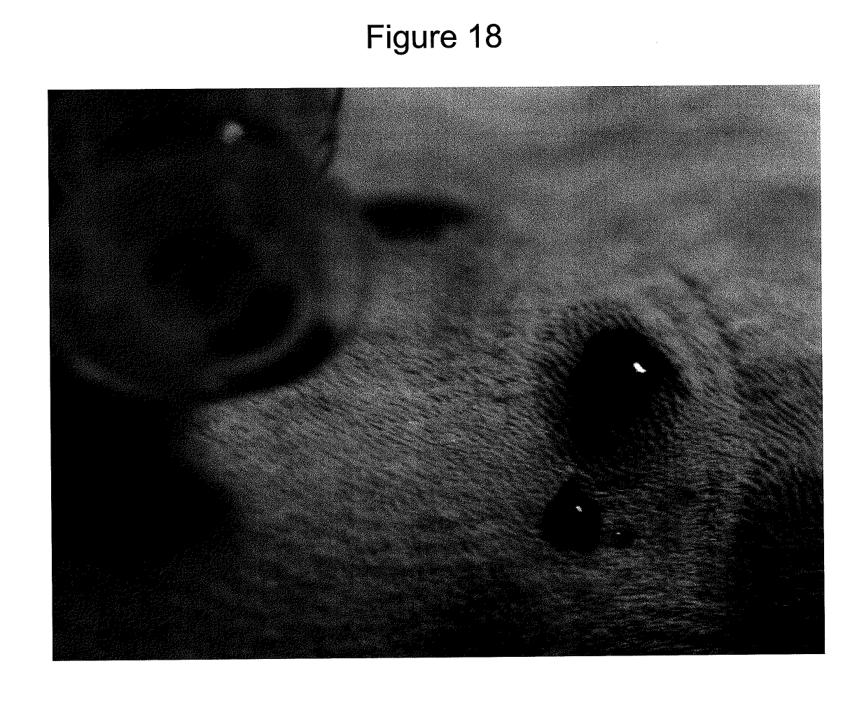

Other studies were conducted in rats using the intradermal device delivering a test agent, Indian ink. As can be seen in FIGS. 14-18, the delivery of the test agent was limited to just the dermal layer of the rat skin. FIG. 16 shows the results of delivery of two volumes (0.1 cc and 0.2 cc) of the test agent.

### Example 5

### Intradermal Delivery in Live Humans

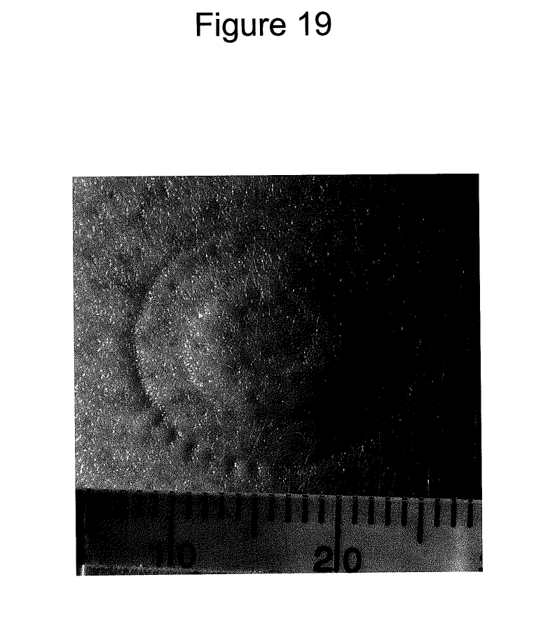

One advantage of the present invention is its ability to delivery an agent to the intradermal layer with minimal pain or scarring at the site of injection. FIG. 19 is a photograph of the area of injection of a live human. As can be seen in the photograph, there is no bleeding or scarring at the injection site and the test subject reported minimal sensation for the injection.

### Example 6

### Intradermal Delivery in Pigs

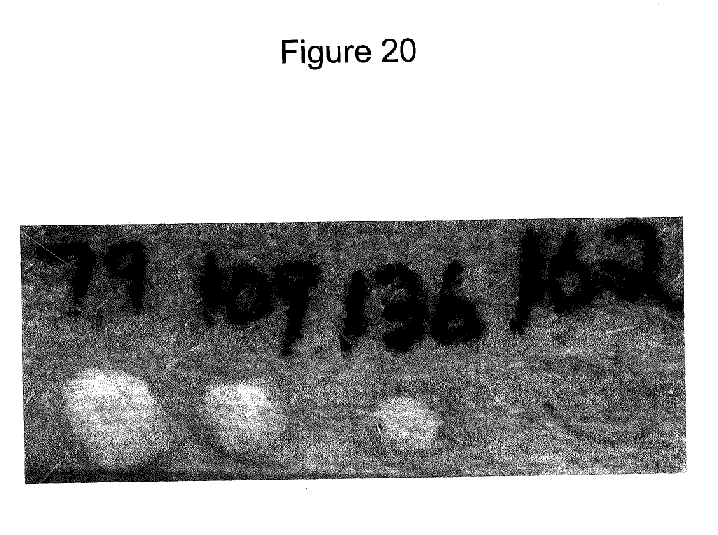

Testing was conducted in pigs to demonstrate that the force of the spring (i.e., spring energy) can be titrated to achieve certain types of delivery. As shown in FIG. 20, the right-most injection is completely SC and the left most is completely ID. The intervening injections are somewhere in between ID and SC. This result shows that the Applicant have control over the distribution of the injection and can place the agent at the required level to achieve the desired results.

1. A needle-free intradermal injection device that is capable of delivering a volume of about 0.05 cc to about 0.5 cc of an agent of interest to the intradermal space of an individual using a spring with a spring force of about 35 to about 130 pounds, wherein the delivery of the agent is through an orifice of about 0.007 inches.

2. The intradermal injection device of claim 1 wherein the orifice material comprises polypropylene.

3. The intradermal injection device of claim 1 or 2 wherein the volume of the agent is 0.1 cc or 0.2 cc.

4. The intradermal injection device of claim 1 wherein the delivery of an agent to an individual has an injection time of less than about 1 second.

5. (canceled)

6. The intradermal injection device of claim 1 wherein the spring force is about 50 pounds to about 100 pounds.

7. (canceled)

8. A method of eliciting an immune response in an individual comprising using the device of claim 1 to administer an effective amount of an agent of interest to the intradermal layer of the individual.

9. The method of claim 8 wherein the immune response is activating dendritic cells and/or antigen presenting cells.

10. The method of claim 8 wherein about 75% of the agent is administered to the intradermal layer.

11. The method of claim 8 wherein about 50% of the agent is administered to the intradermal layer.
